(19) United States
(12) Patent Application Publication
(10) Pub. No.: US 2006/0183114 A1
(43) Pub. Date: Aug. 17, 2006

(54) Novel Eosinophil Cationic Proteins used in the Treatement of Asthematic and Allergic Disorders

(76) Inventors: Erik Hogbom, Uppsala (SE); Jonas Bystrom, Uppsala (SE); Ulla-Britt Jonsson, Uppsala (SE); Per Venge, Uppsala (SE)

Correspondence Address: FISH & RICHARDSON P.C., PO BOX 1022, MINNEAPOLIS, MN, 55440-1022, US

(21) Appl. No.: 10/275,658

(22) PCT Filed: May 8, 2001
(86) PCT No.: PCT/IB01/00927

§ 371 Date: Jun. 11, 2003

(30) Foreign Application Priority Data
May 9, 2000 (CH) 0001706-1

(51) Int. Cl. C12Q 1/68; G01N 33/53; G01N 33/567; G01N 33/574; G01N 33/554; G01N 33/569; C07H 21/04; C07K 14/47

(52) U.S. Cl. 435/6; 435/7.2; 435/7.23; 435/7.32; 435/69.1; 435/320.1; 435/325; 530/350; 536/23.2

(57) ABSTRACT

The present invention relates to novel eosinophil cationic proteins characterised by the presence of a single nucleotide polymorphism. The presence of such polymorphism has been correlated to an advantageous phenotype being a reduced disposition to allergic and asthmatic disorders.

The present invention relates to novel proteins and nucleotide sequences encoding said peptides, pharmaceutical compositions containing or targeting said peptides or nucleotide sequences, assays utilising said peptides or nucleotide sequences and methods of detecting the presence or absence of said proteins and polynucleotide sequences encoding them.

Eosinophil cationic protein (ECP) is a cationic toxin stored in the large specific granules of human eosinophilic leukocytes. It is believed to be a member of the ribonuclease family (Slifman N R et al. J Immunol. (1986) 137, 2193-2917) now designated as RNASE 3.

Background teaching on ECP have been presented by Victor A McKusick in “Online Mendelian Inheritance in Man (OMIM)”, John Hopkins University, Baltimore, Md. MIM Number 131398 (last edited 16 Sep. 1997) on www.ncbi.lm.nih.gov/Omim:
“Eosinophil cationic protein, like eosinophil-derived neurotoxin, is localized to the granule matrix of the eosinophil. Both proteins possess neurotoxic, helminthotoxic, and ribonucleolytic activities. The cDNA sequences of the genes encoding these 2 proteins and the, deduced amino acid sequences indicate that the genes belong to the ribonuclease gene superfamily. Hamann et al. (Genomics 7: 535-546, 1990) demonstrated remarkable similarities of eosinophil-derived neurotoxin and eosinophil cationic protein, symbolized RNS2 and RNS3, respectively, in noncoding sequences, introns, and flanking regions, as well as the previously known coding regions. A single intron in the 5-prime untranslated region and an intronless coding region appear to be features common to many members of the RNase gene superfamily. Both genes were mapped to 14q24-q31 by a combination of analysis of somatic cell hybrids and in situ hybridization (Hamann et al., 1990 supra.). Mastrianni et al. (Genomics 13: 240-242, 1992) confirmed the assignment of both RNS2 and RNS3 to chromosome 14 by Southern analysis of DNAs from mouse-human cell hybrids. They commented on the fact that another eosinophil granule protein is encoded by a gene on chromosome 19 and yet another, eosinophil peroxidase, by a gene on chromosome 17.”

Some heterogenicity has been noted in the molecular weight of ECP and this is believed to be explained by difference in the pattern of glycosylation of the protein. The ribonuclease and anti-bacterial effects of ECP have been investigated on a genotypic level by Rosenberg HF (J Biol Chem (1995) 270, 7876-7881) who by comparing wild-type and mutant ribonuclease defective recombinant ECP has shown that the mutant ribonuclease defective form retained anti-bacterial activity. The two mutant forms were generated by single base pair conversions at positions 517 (C to G resulting in the amino acid conversion His 128 to Asp) and 248 (A to G resulting in the amino acid conversion Lys38 to Arg).

It has now been observed that of the different ECP's described in the art, a specific class of ECP bears a genotypic variation which is believed to be phenotypically advantageous. The present invention relates to such ECP proteins, as will be defined hereinafter, together with nucleotide sequences encoding said proteins.

The genotypic difference that characterises the proteins of the present invention, arises from a single nucleotide polymorphism at position 926 of the nucleotide sequence for ECP given as Genbank accession number X16545 (SEQ ID No. 1). This results in replacement of arginine residue at position 97 in the expressed ECP amino acid sequence by a threonine residue.

Thus the present invention relates to the use of a protein selected from;

(a) an ECP,

(b) a mutant of said ECP, and

(c) a fragment of (a) or (b),

said protein lacking an arginine 97 of wild-type ECP or its equivalent, in the preparation of a medicament for the prevention and/or treatment of allergic or asthmatic disorders.

Thus, the ECP for use in the present invention may comprise a modification at amino acid residue 97 or its equivalent such that residue 97 or its equivalent is any other amino acid other than arginine.

The term “wild-type ECP (WT-ECP)” is used to refer to the protein sequence expressed by the DNA sequence shown herein as SEQ ID No. 1, having an arginine residue at position 97 (Arg97) and shown herein as SEQ ID No. 2. All reference to ECP protein of SEQ ID No. 2 or as expressed by SEQ ID No. 1 refer to the sequence of mature protein i.e. excluding the signal peptide. The relevant regions of the polynucleotide/protein sequences are identified in the sequence listing below.

The term “an ECP” as used herein in terms of the present invention, means an ECP protein wherein at least the amino acid residue corresponding to Arg97 of wild type ECP is modified.

A “mutant of said (an) ECP” is used to mean any variant or homologue of the ECP of the present invention, such variants and homologues being defined below.

The term “an ECP of the present invention” is used to include options (a), (b) and (c) given above.

Any of the above terms followed by “encoding polynucleotide” means a polynucleotide sequence capable of encoding the said ECP.

It has been observed that the presence of an ECP of the present invention in a mammal, or most preferably a human, provides certain advantages such as protection, either partial or preferably complete protection against allergic and/or asthmatic disorders and its absence indicates a predisposition to such disorders. Thus, the presence of such an ECP encoding polynucleotide in heterozygous presentation has been observed to provide partial protection, whereas, its presence homozygously has been observed to provide complete protection. Such disorders include asthma, excema and rhinitis,

As discussed above, the invention is based upon the observation that a single base mutation giving rise to an amino acid replacement at position 97 of wild type ECP is advantageous. For example, in a preferred embodiment the ECP of the invention comprises any amino acid other than arginine, preferably threonine or a biostere thereof, at position 97 in place of arginine. In a further preferred embodiment, (discussed in detail below) an ECP of the present invention may comprise the sequence C T Y, corresponding to residues 96-98 of wild-type ECP or a biostere thereof. The term “biostere” is used as understood in the art, but for example, such biosteres will be of the formula C X₁ Y, wherein X₁ is any amino acid residue other than arginine.

A further aspect of the invention relates to a protein selected from;

(a) an ECP,

(b) a mutant of said ECP, and

(c) a fragment of (a) or (b),

wherein arginine 97 of wild-type ECP, or its equivalent, is replaced by any amino acid other than valine, glycine or lysine.

The present invention further relates to polynucleotides, preferably isolated DNA, encoding a protein of the present invention, nucleotide vectors containing said polynucleotides and host cells containing said polynucleotides or vectors, all as discussed hereinafter.

An additional aspect of the present invention relates to assays for the identification of compounds capable of mimicing an ECP of the present invention or alternatively antagonising the allergy and or asthma inducing effect of wild-type ECP. This aspect further includes the compounds identified by such assays.

Studies carried out in patients suffering from allergic and asthmatic disorders have shown that while wild-type ECP is highly expressed in sufferers, cellular expression of ECP is restricted in heterozygous individuals or individuals homozygous for the ECP mutation 97 of the invention. Thus, reducing cellular levels of wild-type ECP, or preventing expression of wild-type ECP all together, may be used in the prevention of allergic and/or asthmatic disorders. The present invention therefore also provides a method of reducing or preventing expression of ECP. In an alternative embodiment the present invention provides a method of inhibiting ECP activity, preferably within the cell. In particular, the present invention provides a method of inhibiting wild-type ECP RNase activity. Thus, the present invention also provides antagonists of ECP capable of reducing or inhibiting ECP expression and/or activity. As such, there is also provided assays for the identification of ECP antagonists. ECP antagonists may include for example the proteins or nucleic acids of the present invention insofar as they can be used as antagonists of wild-type ECP, in gene therapy or antisense therapy.

A further aspect of this invention relates to a method for detecting individuals having a predisposition or susceptibility to certain disease states, in particular, allergic or asthmatic disorders. It is a further aspect of the invention to identify individuals having such a predisposition or susceptibility by identifying those individuals with an altered WT-ECP encoding polynucleotide.

Accordingly, the invention provides a method of diagnosis comprising determining whether an individual is homozygous or heterozygous for a ECP encoding polynucleotide and a polymorphism thereof. The method comprises screening for an individual at risk of a condition or disease correlated with presence of the polymorphism.

This aspect of the invention further extends to the use of the polynucleotides and polypeptides of the present invention in the treatment of such a patient with a mimetic of an ECP of the invention or an agent capable of inducing a genotypic modification to give rise to the expression of an ECP of the invention.

The preferred embodiments of the present invention will be discussed below under the appropriate headings;

Protein

The term “protein” includes polypeptides having at least more than 5, 10 or 20 amino acids.

The ECP proteins of use in the present invention all include replacement of arginine at position 97 of WT-ECP by an alternative amino acid residue. Preferably the replacement is effected by a threonine residue or biostere thereof at the position corresponding to Arg97 of WT-ECP. Preferably, such proteins comprise the sequence C T Y or more preferably N C T Y A or biosteres thereof at positions corresponding to amino acids 96-98 or 95-99 of WT-ECP respectively. The term biostere is used as understood in the art and encompasses modifications made to this sequence by; (a) one or more amino acid residues being replaced by a naturally or non-naturally occurring amino acid residue (b) the order of two or more amino acid residues being reversed, (c) both (a) and (b) being present together and (d) a spacer group being present between any two amino acid residues, provided the resultant protein retains the activity of the parent protein.

The remaining protein sequence may be identical to WT-ECP or a variant, homologue or derivative thereof, herein all defined as mutant ECP's. Such variant, homologue or derivative forms are discussed in detail below.

A most preferred ECP of the present invention relates to that given as SEQ ID No. 4.

The following description of polypeptide homologues, variants and derivatives is to be read in conjunction with the ECP's of the invention described above and each preferred embodiment described.

Polypeptide Homologues

It will be understood that protein sequences of the invention or for use in the invention are not limited to the particular sequences or fragments thereof or sequences obtained from the particular protein but also include homologous sequences obtained from any source, for example related viral/bacterial proteins, cellular homologues and synthetic peptides, as well as variants or derivatives thereof

Thus, the present invention covers variants, homologues or derivatives of protein sequences of the present invention, as well as variants, homologues or derivatives of the nucleotide sequence coding for the protein sequences of the present invention.

In the context of the present invention, a homologous sequence is taken to include an amino acid sequence which is at least 60, 70, 80 or 90% identical, preferably at least 95 or 98% identical at the amino acid level over at least 15 residues including the modified residue corresponding to arginine 97, preferably from 15 to 50 amino acids that include said modified residue. In particular, homology should typically be considered with respect to those regions of the sequence known to be essential for providing protection from asthmatic or allergic disorders rather than non-essential neighbouring sequences. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an. “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed.

Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and PASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Polypeptide Variants and Derivatives

The terms “variant” or “derivative” in relation to the amino acid sequences of the present invention includes any substitution of, variation of, modification of, replacement of, deletion of or addition of one (or more) amino acids from or to the sequence providing the resultant amino acid sequence retains the advantageous properties of the parent ECP protein as described above.

An ECP of the invention may be modified for use in the present invention. Typically, modifications are made that maintain the protection providing property of the sequence. Amino acid substitutions may be made, for example from 1, 2 or 3 to 10, 20 or 30 substitutions provided that the modified sequence retains such properties. Amino acid substitutions may include the use of non-naturally occurring analogues, for example to increase blood plasma half-life of a therapeutically administered polypeptide.

Thus, homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid (hereinafter referred to as B), norleucine (hereinafter referred to as O), pyriylalanine, thienylalanine, naphthylalanine and phenylglycine, a more detailed list of which appears below.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar - charged | D E |
| | | K R |
| AROMATIC | | H F W Y |

Such replacements may also be made by unnatural amino acids include; alpha* and alpha-disubstituted* amino acids, N-alkyl amino acids*, lactic acid*, halide derivatives of natural amino acids such as tifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid#, 7-amino heptanoic acid*, L-methionine sulfone#*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid# and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

Proteins of the invention are typically made by recombinant means, for example as described below. However they may also be made by synthetic means using techniques well known to skilled persons such as solid phase synthesis. Proteins of the invention may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the function of the protein of interest sequence.

Proteins of the invention may be in a substantially isolated form. It will be understood that the protein may be mixed with carriers or diluents which will not interfere with the intended purpose of the protein and still be regarded as substantially isolated. A protein of the invention may also be in a substantially purified form, in which case it will generally comprise the protein in a preparation in which more than 90%, e.g. 95%, 98% or 99% of the protein in the preparation is a protein of the invention.

Polynucleotides

An aspect of the invention provides a polynucleotide capable of encoding any of the above defined ECP polypeptides, or a fragment thereof. In a preferred embodiment, the polynucleotide is an ECP encoding polynucleotide in which guanosine at position 926 is replaced with cytosine. A fragment of such a polynucleotide comprises position 926 and is at least 15 nucleotides in length. Preferably, the polynucleotide is an isolated DNA molecule which means that it is free from other DNA molecules that are naturally associated therewith in nature. Such a polynucleotide is preferably capable of expressing an ECP protein of the invention as hereinbefore described.

As discussed above, the present invention is based upon the observation of a single base polymorphism at position 926 of a human ECP encoding gene. A further aspect of this observation is that the polymorphism is correlated with a predisposition to a an allergic or asthmatic disorder. The invention is of advantage in that by screening for the presence of the polymorphism it is possible to identify individuals likely to have this predisposition.

Polynucleotides of the invention or for use in the invention comprise nucleic acid sequences encoding the polypeptide sequences of the invention. Such polynucleotides may be identified on the basis of a change in the Pst I restriction digest pattern of said sequence when compared to that of a WT-ECP encoding polynucleotide (WT-ECP-DNA, for example SEQ ID No. 1). Thus, Pst I digestion of WT-ECP-DNA gives rise to 2 sub-fragments by virtue of a Pst I site at bases 921 to 926. Polynucleotide sequences encoding an ECP of the present invention lack the corresponding Pst I site and a single fragment will be obtained. This difference may also be utilised in the methods of detecting the presence or absence of an ECP encoding sequence as discussed below.

As the preferred proteins of the present invention include a threonine residue at the position corresponding to Arg97 in WT-ECP, or in more preferred embodiments the sequences C T Y or N C T Y A, the polynucleotide sequences encoding such proteins will at least include the nucleotide sequence ACG or in preferred embodiments (AAC) TGC ACG TAT (GCA) corresponding to bases (919) 922 to 930 (933) of WT-ECP-DNA. This particular region may be varied within the confines of the definition of the term “or a biostere thereof” as discussed above. The remaining polynucleotide may be identical to WT-ECP-DNA or a variant, homologue or derivative thereof as discussed in detail below.

A specific embodiment of the invention is the nucleotide sequence of SEQ ID NO: 3, listed in the sequence listing below. In SEQ ID NO: 3, the polymorphism lies in C in place of G at position 926 of SEQ ID NO: 1 (WT-ECP).

It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

Polynucleotides of the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides of the invention,

The terms “variant”, “homologue” or “derivative” in relation to the nucleotide sequences include any substitution of, variation of, modification of, replacement of, deletion of or addition of one (or more) nucleic acid from or to the sequence providing the resultant nucleotide sequence codes for a polypeptide retaining the advantageous characteristics of the ECP proteins of the present invention, preferably having at least the same activity as sequences presented in the sequence listings.

As indicated above, with respect to sequence homology, preferably there is at least 75%, more preferably at least 85%, more preferably at least 90% homology to the sequences shown in the sequence listing herein. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides of the invention capable of selectively hybridising to the nucleotide sequences presented herein, or to their complement, will be generally at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred polynucleotides of the invention will comprise regions homologous to nucleotides that include the Pst I site discussed above.

The term “selectively hybridizable” means that the polynucleotide used as a probe is used under conditions where a target polynucleotide of the invention is found to hybridize to the probe at a level significantly above background. The background hybridization may occur because of other polynucleotides present, for example, in the cDNA or genomic DNA library being screening. In his event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC{1×SSC=0.15 MNaCl, 0.015 M Na₃ Citrate pH 7.0).

Where the polynucleotide of the invention is double-stranded, both strands of the duplex, either individually or in combination, are encompassed by the present invention. Where the polynucleotide is single-stranded, it is to be understood that the complementary sequence of that polynucleotide is also included within the scope of the present invention.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of SEQ I.D. Nos 1-6 under conditions of medium to high stringency. Similar considerations apply to obtaining species homologues and allelic variants of the polypeptide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences, such as SEQ ID. No 3. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

Polynucleotides of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as a DNA polynucleotides and probes according to the invention may be produced recombinantly, synthetically, or by any means available to those of skill in the art. They may also be cloned by standard techniques. An example of how ECP encoding polynucleotides, such as those of the present invention may be produced recombinantly is provided in Rosenberg H, J Biol Chem (1995) 270, 7876-7881.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g. of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Nucleotide Vectors

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Vectors of the invention may be transformed or transfected into a suitable host cell as described below to provide for expression of a protein of the invention. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Control sequences operably linked to sequences encoding the protein of the invention include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

The promoter is typically selected from promoters which are functional in mammalian, cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyruvate kinase). They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

Host Cells

Vectors and polynucleotides of the invention may be introduced into host cells for the purpose of replicating the vectors/polynucleotides and/or expressing the proteins of the invention encoded by the polynucleotides of the invention. Although the proteins of the invention may be produced using prokaryotic cells as host cells, it is preferred to use eukaryotic cells, for example yeast, insect or mammalian cells, in particular insect cells such as those including a polyhedrin promoter.

Vectors/polynucleotides of the invention may introduced into suitable host cells using a variety of techniques known in the art, such as transfection, transformation and electroporation. Where vectors/polynucleotides of the invention are to be administered to animals, several techniques are known in the art, for example infection with recombinant viral vectors such as retroviruses, herpes simplex viruses and adenoviruses, direct injection of nucleic acids and biolistic transformation.

Protein Expression and Purification

Host cells comprising polynucleotides of the invention may be used to express proteins of the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins of the invention can be extracted from host cells by a variety of techniques known in the art, including enzymatic, chemical and/or osmotic lysis and physical disruption. Rosenberg H (supra) describes how ECP proteins may be produced recombinantly.

Administration

Proteins of the invention and substances identified or identifiable by the assay methods of the invention may preferably be combined with various components to produce compositions of the invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. The composition of the invention may be administered by direct injection. The composition may be formulated for parenteral, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

Polynucleotides/vectors encoding polypeptide components for use in affecting viral infections may be administered directly as a naked nucleic acid construct, preferably further comprising flanking sequences homologous to the host cell genome. When the polynucleotides/vectors are administered as a naked nucleic acid, the amount of nucleic acid administered may typically be in the range of from 1 μg to 10 mg, preferably from 100 μg to 1 mg.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Preferably the polynucleotide or vector of the invention is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, intraocular or transdermal administration.

The routes of administration and dosages described are intended only as a guide since a skilled practitioner will be able to determine readily the optimum route of administration and dosage for any particular patient and condition.

Assays

The invention further includes assay, being methods of detecting the presence or absence of a polymorphism in an ECP-encoding polynucleotide. In this embodiment of the invention, the method of detection may employ a polymerase chain reaction, single strand conformational polymorphism assay, or any such detection technique described below under the heading “Genotyping”, and determining whether an individual possesses a wild type ECP encoding polynucleotide or a polymorphism thereof. Each individual may be homozygous for the wild type, heterozygous for the wild type and a polymorphism, or homozygous for polymorphisms in the ECP encoding polynucleotide. Presence of a polymorphism correlates with predisposition to an allergic or asthmatic disorder. Optionally, the method further comprises use an indicator means to react to the presence of the polymorphism. In this respect the term “polymorphism” is used to refer to that that distinguishes the ECP proteins/polynucleotides of the present invention from those described herein as wild-type.

Indicator means typically induces a detectable signal upon presence of the polymorphism, and can induce a colour change or a coagulation or induce a restriction site, detectable by further analytical steps. Another indicator means comprises an antibody that has binding affinity that distinguishes between a wild type sequence and a polymorphism.

A particular method of the invention comprises screening for a polymorphism in an ECP encoding polynucleotide by virtue of the absence of a Pst I restriction site at bases 921 to 926 of the ECP DNA sequence shown in SEQ ID No. 1, wherein presence of the polymorphism correlates with predisposition to an allergic or asthmatic disorder. This method is discussed above in respect of the polynucleotides of the present invention.

In use of a specific embodiment of the invention to be described below in further detail, an individual is screened to determine whether he or she possess a Pst I restriction site at bases 921 to 926 of an ECP encoding polynucleotide which is a published sequence or is a polymorphism thereof in which a guanosine nucleotide at position has been replaced by an cytosine nucleotide. In this specific embodiment, the presence of the polymorphism in which guanosine is replaced by cytosine at position 926 correlates with a predisposition to an allergic or asthmatic disorder.

Screening is carried out, for example, using PCR primers adapted to amplify a portion of an ECP encoding polynucleotide that includes the nucleotide at position 926. It is preferred that the PCR primers are selected so as to amplify a region of the polynucleotide that surrounds position 926 and includes at least six nucleotides on either side of this position. Such a pair of primers are shown herein as SEQ ID's Nos. 5 and 6. These primers will give rise to a 644 bp fragment of SEQ ID No. 1 as described in Example 1. This fragment includes the region encoding the ECP protein. PCR techniques are well known in the art and it would be within the ambit of a person of ordinary skill in this art to identify primers for amplifying a suitable section of the ECP encoding polynucleotide that includes the nucleotide at position 926. PCR techniques are described for example in EP-A-0200362, EP-A-0201184, and U.S. Pat. Nos. 4,683,195, 4,683,202 and 4, 965,188. The amplified products may then be subjected to Pst I digestions and the resultant fragments separated by, for example gel electrophoresis. The pattern of fragments produced by such Pst I digestion is indicative as to the presence of absence of an ECP encoding sequence of the present invention. Thus, two fragments indicates the presence of WT-ECP encoding DNA, a single fragment indicative of homozygous presentation of the polymorphism at position 926 (as discussed above) and three fragments indicative of heterozygous presentation of the polymorphism.

In a further embodiment of the invention, the diagnostic method comprises analysis of the region surrounding position 926 of an ECP encoding polynucleotide using single strand conformational polymorphism (SSCP) mapping. It is preferred that the PCR primers for this purpose are selected so as to be homologous with a region of the genome within 200 bp of position 926 on the ECP encoding polynucleotide. It is further preferred that the PCR primers are selected so that position 926 is substantially towards the middle of the amplified DNA segment.

The invention further provides a diagnostic kit comprising diagnostic means according to this aspect of the invention, optionally within a container. Thus, the invention further provides a diagnostic kit comprising a carrier means such as a carton or box being compartmentalised to receive in close confinement therein the detection means according to the invention, optionally within a container means such as a vial, tube, ampoule, and the like. Further container means may also be present which comprise other elements of the method of detection as described herein.

The detection means of the present invention may preferably comprise primers SEQ ID NO:s 5 and 6, the sequences of which are shown in the sequence listing below. The 5′ end of SEQ ID NO: 5 binds at position 495 of SEQ ID No. 1, the 3′ end of SEQ ID NO: 6 binds at position 1114. Preparation of further primers suitable for determining genotype of a ECP encoding polynucleotide will be within the ambit of a person of ordinary skill in the art.

Further aspects of the present invention relate to assays for compounds capable of acting as agonists of an ECP protein of the present invention. Such agonists may be administered to patients identified by a method of detection described above as having a predisposition to an allergic or asthmatic disorder. While not wishing to be bound by theory, it is believed that the ECP proteins of the present invention provide their beneficial properties by not being cytotoxic and/or by not being fibroblast activating like wild-type ECP. Assays that are capable of measuring whether these proposed end-points, particularly fibroblast activation, may be of use in identifying agonists of the ECP proteins described herein. The mutant ECPs described above are examples of such agonists.

End point assays of the present invention, used to identify mimics or agonists of the ECP proteins described herein include, but are not limited to:

1. Assays for Detecting Cytotoxic Properties with Respect to the Lilling of Bacteria and/or Cancer Cells;

FMCA procedure: A modification of the fluorometric microculture cytotoxic assay (FMCA) described by Larson R et al (1992) is used. An erythroleukemic K562 cell line is cultured in RPMI 1640 (HyClone, Cramlington, UK) supplemented with 10% heat inactivated foetal calf serum (FCS) (HyClone, Cramlington, UK), penicillin 60 μg/mL and streptomycin 50 μg/mL (HyClone, Cramlington, UK). By the day of assay, the cells are washed three times in RPMI 1640 supplemented with penicillin 60 μg/mL and streptomycin 50 μg/mL and without FCS. K562 cells, 20 000 cells/well, are seeded into wells of V-shaped 96-well microtiter plates (Nunc, Roskilde, Denmark) in triplicates, 10 μL ECP in 0.2 M Na-acetate buffer pH 5.5 at a final concentration of 20 μg/mL/well is added. Wells with cells and buffer serve as a negative control. As a positive control for cytotoxicity, cells treated with Triton at a final concentration of 0.01 % are employed. The culture plates are then incubated at +37° C. in humidified atmosphere containing 95% air and 5 % CO₂ for 72 hr, followed by centrifugation (200×G, 7 min). After medium removal and one wash with PBS 200 μL/well, 100 μL/well of PBS containing fluorescein diacetate (FDA) (Sigma Chemical, Co, St. Louis, Mo., USA) (10 μg/mL) is added. Subsequently the plates are incubated for 1 hr at +37° C. before reading fluorescence with filters set at 485 and 538 for excitation and emission, respectively (Fluorescan 2, Labsystems OY, Helsinki, Finland). The fluorometer is blanked against wells containing PBS including FDA dye but without cells. The fluorescence data is transferred to custom-made software for automated data calculation using Microsoft Excel and a McIntosh SE/30 personal computer. The results obtained by the indicator FDA are presented as survival index (SI) defined as fluorescence in test wells in percent of control wells. Individual column fractions were tested for toxicity to K562 cells. Quality criteria for a successful assay include a fluorescence signal in control cultures of >5×mean blank values and mean coefficient of variation in control wells of <20%.

DiSC procedure: In parallel, a modified form of the short-term dye-exclusion test, Differential Staining and Cytotoxicity (DiSC) assay (Weisenthal L M, Marsden J A, Dill P L, Macaluso C K. A Novel dye exclusion method for testing in vitro chemosensitivity of human tumors. Cancer Res 1983, 43:749-57, Nygren P, Kristensen J, Jonsson B, Sunstrom C, Lonnerholm G, Kreuger A, Larsson R. Feasability of the fluorometric microculture cytotoxocitity assay (FMCA) for cytotoxic drug sensitivity testing of tumor cells from patients with acute lymphoblastic leukemia. Leukemia 1992 6:1121-8) is performed. Immediately after fluorescence measurement, selected wells are exposed to a mixture of Fast green, 1%, Nigrosin, 0.5% (Sigma Chemical, Co, St Louis, Mo., USA) and 25 000 formaldehyde-fixed chicken elythrocytes/well for 10 min at room temperature. The cellular content of the wells is subsequently cytocentrifuged onto slides using a Cytospin 3 (Shandon, Astmoore, UK) and counter stained with May-Grunewald-Giemsa stain. Cell survival is evaluated by light microscopy. Viable cells stain normal Giemsa morphology, whereas dead cells and chicken erythrocytes stain greenish-black. Tumour cell survival using this modified DiSC procedure (Nygren et al 1992-supra) is calculated by expressing the survival index (SI) as a ratio of viable K562 cells to fixed erythrocytes in experimental wells as a percentage of the ratio obtained in control wells.

2. Assays for Detecting RNase Activity;

Rapid RNase detection may be achieved using a cleavable fluorescent-labelled RNase substrate. 5 μl of 10× RnaseAlert Buffer (RnaseAlert Kit-Ambion and Integrated DNA Technologies, Inc.) is pipeted into tubes containing lyophilized Fluorescent Substrate. Up to 45 μl of the solution to be tested is then added and the mixture is incubated for 30-60 minutes at 37° C. The Fluorescent Substrate is a modified RNA oligonucleotide that emits a green fluorescence if it is cleaved by RNase. The fluorescence is visually detected using short-wave UV illumination or measured in a fluorometer. Solutions containing RNase activity will produce a green glow in the assay whereas solutions without RNase activity will not fluoresce. The amount of RNase activity will be directly proportional to the rate of fluorescence increase. Quantitative measurements can be obtained from a fluorometer.

3. Assays for Measuring the Capacity of a Compound to Affect Fibroblasts in their Production of Collagen and Other Proteoglycans;

Cultures of human embryonic lung fibroblasts (EL-1) are established according to Malmström (Malmström A, Fransson L A Biosynthesis of dermatan sulfate. I. Formation of L-iduronic acid residues. J Biol Chem 1975, 250:3419-25), cultivated in 25 cm² cell culture flasks, 24-well cell culture plates or 96-well microplates in Dulbecco's modified Eagle's medium with 10% new-born calf serum (NCS) and grown to confluence. At confluence, the medium is changed to a sulfate poor medium (Dulbecco's special medium) containing 0.4% NCS, supplemented with 50 mg/ml ascorbic acid, 0.2 mM L-prolin and 4 mM glutamine. The cultures are then reconditioned for 3-4 hours. ECP is added to the cultures in different concentrations (0.1, 1, 10, 100 μg/ml Dulbecco's special medium. 0.4 ml/well). Medium alone with 0.4% NCS served as control. After 24 hours the radioactive precursor ([³⁵S]-sulphate, 200 μCi/ml) is added for an additional 24 hours. The medium is then decanted and the remaining cell layer is washed with phosphate-saline buffer that also are added to the medium fractions. The cell layer is further extracted with 4 M guanidine chloride, 0.05 M sodium acetate, pH 5.8 containing protease inhibitors (0.01 M EDTA, 0.005 M N-ethylmalemide) and 1% triton X-100 overnight. Both mediLun and cell extract (the latter after dilution with 20 volumes of 6 M urea, 0.05 M sodium acetate, pH 5.8 containing the same protease inhibitors as above, 5 μg ovalbumin and 0.1% triton X-100) are subjected on columns (0.5×0.7 cm) of DE-52. All other experiments with ECP used a concentration of 10 μg/ml.

Proteoglycans are precipitated with Alcian blue (Bjornsson S Simultaneous preparation and quantitation of proteoglycans by precipitation with alcian blue. Anal Biochem 1993 210:282-91). The methods specificity is based on low pH and high salt concentration in combination with detergent. The Alcian blue precipitation is used for quantitation of proteoglycans and glycosaminoglycans. The proteoglycans are further analysed before and after digestion with electrophoresis (Bjornsson S Size-dependent separation of proteoglycans by electrophoresis in gels of pure agarose. Anal Biochem 1993 210:292-8). The electrophoresis method used is a discontinuous buffer system. The cathode (over) buffer is 0.1 M Tris-Ac buffer and in the anode buffer the Tris-Ac is decreased to 0.01 M, pH 7.3. All samples are added to a sample gel of tris-glycine (TG)×2, 2.5 % SDS, 0.1% agarose, and 15% glycerol. These samples are applied to a gel of 2% agarose. The gels are analysed and scanned on the Fuji bio image analyse system from Fuji Photo Film CO., Ltd.

Culture medium combined with PBS washes, are precipitated by addition of ethanol to a final concentration of 67% at 40° C. overnight. Protein is separated from free amino acids by filtration through a 0.45 μm pore filter (type HV) using a vacuum filtration unit. The supernatant is retained and the filter washed twice with ethanol (67%). The filter with adherent proteins is hydrolysed in hydrochloric acid (HCl, 6 M) at 110° C. for 16 hours. Supernatants are evaporated to dryness and hydrolysed as above. Hydrolysates are mixed with charcoal (30 mg) and filtered (0.65 μm, type DA) prior to chromatography. Hydroxyproline is isolated and measured by reveresed-phase-HPLC after derivatization with 7-cloro-4-nitorbenzo-2-oxa-1,3-diazole (NBD-Cl). Briefly, a 200 μl aliquot of the hydrolysates prepared as described above is buffered with potassium tetraborates (100 μl, 0.4 M) and reacted with 12 MM NBD-Cl in methanol (100 μl). Samples are protected from light with aluminium foil and incubated at 37° C. for 20 minutes. The reaction is stopped by addition of hydrochloric acid (50 μl, 1.5 M) and finally 150 μl sodium acetate (167 mM) in acetonitrile (26% V/V) is added. Samples are filtrated (pore size 0.22 μm, type GV, Millipore, UK) and a 100 μl aliquot is loaded onto the column. The Hydroxyproline content in each sample is determined by comparing peak areas of samples from the chromatogram to those generated from standard solutions, derivatized and separated under identical conditions.

Hydroxyproline measured in the ethanol-insoluble fractions is taken as an index of procellagen production and the rate of procollagen synthesis is obtained from the combined values for ethanol-soluble and combined values for ethanol-soluble and ethanol-insoluble fractions. The DNA from the cell layer are extracted with 500 μl BS over night and then sonicated 10-20 seconds INTENSITET. Bisbenzimidzole is added to samples and the fluorescence spectrophotometer (Perkin-Elmer reader, LS-5B) (Labarca C, Paigen K A simple, rapid, and sensitive DNA assay procedure. Anal Biochem 1980 102:344-52).

The above and other assays may also be used to identify antagonists of wild-type ECP.

These may be identified as agents that are capable of binding to WT-ECP, inhibiting WT-ECP RNase activity, and/or binding to DNA or RNA encoding WT-ECP and hence preventing the induction of allergic and/or asthmatic disorders. Examples of such agents include, but are not limited to, the ECP mutation 97 of the present invention and anti-ECP antibodies such as described in Rosenberg H (supra).

A more detailed discussion of suitable assay techniques is provided below.

Genotyping

As used herein, the term “genotyping” means determining whether a an ECP encoding polynucleotide includes a guanosine at position 926. The term “genotyping” is synonymous with terms such as “genetic testing”, “genetic screening”, “determining or identifying an allele or polymorphism”, “molecular diagnostics” or any other similiar phrase.

Any method capable of distinguishing nucleotide differences in the appropriate sample DNA sequences may also be used. In fact, a number of known different methods are suitable for use in genotyping (that is, determining the genotype) for a an ECP encoding polynucleotide of the present invention. These methods include but are not limited to direct sequencing, PCR-RFLP, ARMS-PCR, Taqman™, Molecular beacons, hybridization to oligonucleotides on DNA chips and arrays, single nucleotide primer extension and oligo ligation assays.

Genotype Screening

In one embodiment, the present invention provides a method for genotype screening of a nucleic acid comprising a an ECP encoding polynucleotide from an individual. The methods for genotype screening of a nucleic acid comprising an ECP encoding polynucleotide from an individual may require amplification of a nucleic acids from a target sample from that individual.

Target Sample

The target samples of the present invention may be any target nucleic acid comprising a an ECP encoding polynucleotide from an individual being analyzed. For assay of such nucleic acids, virtually any biological sample (other than pure red blood cells) is suitable. For example, convenient target samples include but are not limited to whole blood, leukocytes, semen, saliva, tears, urine, faecal material, sweat, buccal, skin and hair. For assay of cDNA or mRNA, the target sample is typically obtained from a cell or organ in which the target nucleic acid is expressed.

Genotyping SNPS

A number of different methods are suitable for use in determining the genotype for an SNP. These methods include but are not limited to direct sequencing, PCR-RFLP, ARMS-PCR, Taqman™, Molecular beacons, hybridization to oligonucleotides on DNA chips and arrays, single nucleotide primer extension and oligo ligation assays. Any method capable of distinguishing single nucleotide differences in the appropriate DNA sequences may also be used.

Amplification

As used herein, the term “amplification means nucleic acid replication involving template specificity. The template specificity relates to a “target sample” or “target sequence” specificity. The target sequences are “targets” in the sense that they are sought to be sorted out from other nucleic acids. Consequently, amplification techniques have been designed primarily for sorting this out. Examples of amplification methods include but are not limited to polymerase chain reaction (PCR), polymerase chain reaction of specific alleles (PASA), ligase chain reaction (LCR), transcription amplification, self-sustained sequence replication and nucleic acid based sequence amplification (NASBA).

Taqman™

Suitable means for determining genotype may be based on the Taqman™ technique. The Taqman™ technique is disclosed in the following U.S. Pat. Nos. 4,683,202; 4,683,195 and 4,965,188. The use of uracil N-glycosylase which is included in Taqman™ allelic discrimination assays is disclosed in U.S. Pat. No. 5,035,996.

PCR

PCR techniques are well known in the art (see for example, EP-A-0200362 and EP-A-0201184 and U.S. Pat. Nos. 4,683,195 and 4,683,202). The process for amplifying the target sequence consists of introducing a large excess of two oligonucleotide primers to the DNA mixture containing the desired target sequence, followed by a precise sequence of thermal cycling in the presence of a DNA polymerase. With PCR, it is possible to amplify a single copy of a specific target sequence in, for example, genomic DNA to a level detectable by several different methodologies (such as hybridisation with a labelled probe, incorporation of biotinylated primers followed by avidin-enzyme conjugate detection and incorporation of ³²P labelled deoxynucleotide triphosphates, such as dCTP or dATP, into the amplified sequence). Alternatively, it is possible to amplify different polymorphic sites (markers) with primers that are differentially labelled and thus can each be detected. One means of analysing multiple markers involves labelling each marker with a different fluorescent probe. The PCR products are then analysed on a fluorescence based automated sequencer. In addition to genomic DNA, any oligonucleotide sequence may be amplified with the appropriate set of primer molecules. In particular, the amplified segments created by the PCR process itself are, themselves, efficient templates for subsequent PCR amplifications. By way of example, PCR can also be used to identify primers for amplifying suitable sections of an ECP encoding polynucleotide in or from a human.

Primers

The present invention also provides a series of useful primers.

As used herein, the term “primer” refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template.

The term “primer site” refers to the area of the target DNA to which a primer hybridizes.

The term “primer pair” means a set of primers including a 5′ upstream primer that hybridizes with the 5′ end of the DNA sequence to be amplified and a 3′ downstream primer that hybridizes with the complement of the 3′ end of the sequence to be amplified.

The primers of the present invention may be DNA or RNA, and single-or double-stranded. Alternatively, the primers may be naturally occurring or synthetic, but are typically prepared by synthetic means.

Primer Hybridisation Conditions

As used herein, the term “hybridisation” refers to the pairing of complementary nucleic acids. Hybridisation and the strength of hybridisation (i.e. the strength of association between the nucleic acids) is impacted by such factors as the degree of complementarity between nucleic acids, stringency of conditions involved, the melting temperature (Tm) of the formed hybrid and the G:C ratio within the nucleic acids.

As used herein, the term “stringency” is used in reference to the conditions of temperature, ionic strength and the presence of other compounds such as organic solvents under which the nucleic acid hybridizations are conducted.

Hybridizations are typically performed under stringent conditions, for example, at a salt concentration of no more than 1M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific primer hybridizations.

Allele Specific Primers

An allele-specific primer hybridises to a site on target DNA overlapping a polymorphism and only primes amplification of an allelic form to which the primer exhibits perfect complementarity (See Gibbs, Nucleic Acid Res. 17, 2427-2448 (1989)). This primer may be used in conjunction with a second primer which hybridises at a distal site. Amplification proceeds from the two primers leading to a detectable product signifying the particular allelic form is present. A control may be performed with a second pair of primers, one of which shows a single base mismatch at the polymorphic site and the other of which exhibits perfect complementarily to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, for example WO 93/22456). Hybridisation probes capable of specific hybridisation to detect a single base mismatch may be designed according to methods known in the art and described in Maniatas et al Molecular Cloning: A Laboratory Manual, 2^{nd} Ed (1989) Cold Spring Harbour.

(i) PCR Primers

Preferably the screening is carried out using PCR primers designed to amplify portions of the human an ECP encoding polynucleotide (gene) that include nucleotide 775.

Examples of such PCR primers are shown as SEQ ID's Nos. 5 and 6.

Detection of Polymorphisms in Amplified Target Sequences

The amplified nucleic acid sequences may be detected using procedures including but not limited to allele-specific probes, tiling arrays, direct sequencing, denaturing gradient gel electrophoresis and single-strand conformation polymorphism (SCCP) analysis.

Allele-Specific Probes

Allele-specific probes can be designed that hybridize to a segment of target DNA from one individual but do not hybridize to the corresponding segment from another individual due to the presence of different polymorphic forms in the respective segments from the two individuals.

As used herein, the term “probe” refers to an oligonucleotide (i.e. a sequence of nucleotides), whether occurring naturally as in a purified restriction digest or produced synthetically, which is capable of hybridizing to another oligonucleotide sequence of interest. Probes are useful in the detection, identification and isolation of particular gene sequences. The hybridisation probes of the present invention are typically oligonucleotides capable of binding in a base-specific manner to a complementary strand of nucleic acid.

The probes of the present invention may be labeled with any “reporter molecule” so that it is detectable in any detection system, including but not limited to enzyme (for example, ELISA, as well as enzyme based histochemical assays), fluorescent, radioactive and luminescent systems. The target sequence of interest (that is, the sequence to be detected) may also be labeled with a reporter molecule. The present invention is not limited to any particular detection system or label.

The hybridization conditions chosen for the probes of the present invention are sufficiently stringent that there is a significant difference in hybridization intensity between alleles, and preferably an essentially binary response, whereby a probe hybridizes to only one of the alleles. The typical hybridization conditions are stringent conditions as set out above for the allele specific primers of the present invention so that a one base pair mismatch may be determined.

Tiling Arrays

The polymorphisms of the present invention may also be identified by hybridisation to nucleic acid arrays, some example of which are described in WO 95/11995. The term “tiling” generally means the synthesis of a defined set of oligonucleotide probes that is made up of a sequence complementary to the sequence to be analysed (the “target sequence”), as well as preselected variations of that sequence. The variations usually include substitution at one or more base positions with one or more nucleotides.

Direct Sequencing

The direct analysis of the sequence of polymorphisms of the present invention may be accomplished using either the dideoxy chain termination method or the Maxam Gilbert method (see Sambrook et al., Molecular Cloning, A Laboratory Manual (2nd Ed., CSHP, New York 1989) or using, for example, Standard ABI sequencing technology using Big Dye Terminator cycle sequencing chemistry analyzed on an ABI Prism 377 DNA sequencer. Preferably., the polymorphism used in the assays of the present invention are identified by the presence or absence of the fragments generated by PstI restriction analysis of the identified sequences.

Denaturing Gradient Gel Electrophoresis

Amplification products of the present invention, which are generated using PCR, may also be analyzed by the use of denaturing gradient gel electrophoresis. Different alleles may be identified based on the different sequence-dependent melting properties and electrophoretic migration of DNA in solution Erlich, ed., PCR Technology, Principles and Applications for DNA Amplification, (W.H. Freeman and Co, New York, 1992), Chapter 7.

Single-Strand Conformation Polymorphism (SCCP) Analysis

Alleles of target sequences of the present invention may also be differentiated using single-strand conformation polymorphism (SCCP) analysis, which identifies base differences by alteration in electrophoretic migration of single stranded PCR products, as described in Orita et al., Proc. Nat. Acad. Sci. 86, 2766-2770(1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products may be related to base-sequence difference between alleles of target sequences.

Identifying Differences Between Test and Control Sequences

These detection procedures for amplified nucleic acid sequences may be used to identify difference of one or more points of variation between a reference and test nucleic acid sequence or to compare different polymorphic forms of the ECP gene from two or more individuals.

Reference Nucleic Acid Sequences

As used herein the term “reference nucleic acid sequence” means a control nucleic acid sequence such as a control DNA sequence representing one or more individuals homozygous for each of the alleles being tested in that assay. By way of example, control DNA sequences may include but are not limited to: (i) a genomic DNA from homozygous individuals; (ii) a PCR product containing a relevant SNP amplified from homozygous individuals; or (iii) a DNA sequence containing a relevant SNP that has been cloned into a plasmid or other suitable vector. The control sample may also be an alleleic ladder comprising a plurality of alleles from known set of alleles. There may be a plurality of control samples, each containing different alleles or sets of alleles. Other reference/control samples typically include diagrammatic representations, written representations, templates or any other means suitable for identifying the presence of a polymorphism in a PCR product or other fragment of nucleic acid. The terms “reference nucleic acid sequence”, reference samples and control samples are used interchangeable throughout the text.

H. Therapeutic Uses

An aspect of the invention provides a screening an individual for a predisposition to an allergic or asthmatic disorder and, if a polynucleotidetic predisposition is identified, treating that individual to delay or reduce or prevent the an allergic or asthmatic disorder.

In an embodiment of this aspect of the invention, the predisposition of an individual to an allergic or asthmatic disorder is assessed by determining whether that individual is homozygous for a ECP encoding polynucleotide in which nucleotide 926 is guanosine, is heterozygous for this polynucleotide and the polymorphism in which guanosine at position 926 is replaced by cytosine, or is homozygous for the polymorphism using methods of detection discussed above.

Thus, an individual who is G/G homozygous at position 926, for the polymorphism is classified as being at highest risk. An individual being GIC heterozygous is classified as having moderate risk. An individual being C/C homozygous is classified as being in the lowest risk category.

Optionally, the assessment of an individual's risk factor is calculated by reference both to the presence of a ECP encoding polynucleotide polymorphism and also to other known polynucleotidetic or physiological or dietary or other indications. The invention in this way provides further information on which measurement of an individual's risk can be based.

General Methodology Reference

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4th Ed, John Wiley & Sons, Inc.

### BRIEF DESCRIPTION OF THE FIGURES

FIG. 1 shows the pattern of Pst I digest fragments of samples containing ECP-encoding DNA indicative the presence/absence of sequences of the present invention.

FIG. 2 shows the RNase activity of wild-type ECP and of ECP mutation 97.

### EXAMPLES

### Example 1

### Identification of SNP in the Human ECP Gene

Material and Methods

Subjects

5 mL EDTA blood was drawn from a mixed population of 70 individuals, medical students and laboratory employees after their written consent

DNA Preparation

200 μL blood were used for DNA preparations as has been described in Kawasaki, 1990, (PCR Protocols, A Guide to Methods and Protocols, Ed. MA Innis pp 146-152, Academic Press, San Diego) with minor modifications. The blood was mixed with 500 μL 10 mM Tris, 0.1 mM EDTA (pH 8.0) and centrifuged for 10 seconds at 15 000 g and the supernatant was discarded. This procedure was repeated three times, until all red blood cells were lyzed. The cell pellet was resuspended in 100 μL Proteinase K-buffer (50 mM KCl, 20 mM Tris-HCl (pH 8.3), 2.5 mM MgCl₂, 0.5% Tween 20, 100 μg/mL Proteinase K) and incubated at 56° C. for 2 hours. Subsequently the samples were heated to 95° C. for 10 minutes to inactivate the proteases. DNA concentration and purity was measured at 260 and 280 nm in a SPECTRAmax™ 250 Microplate Spectrophotometer System (Molecular Devices, USA).

PCR

˜100 ng DNA was used in a 50 μL PCR reaction containing 1.0 U Taq DNA polymerase and buffer from Life Technologies (Gaithersburg, Md.), 1.5 mM MgCl₂, 0.2 μM dNTP, and 20 pmol primers. All base positions refer to Genebank accession number X16545 (SEQ ID No. 1). The biotinylated 3′ primer, 5′-biotin-ggacagttgctgatacccagagtac-3′ (SEQ ID No. 5), between positions 5′-1138 to 3′-1114 and the 5′ primer, 5′-gtgtgtcataaccgagaccggatag-3′ (SEQ ID No. 6), between positions 5′-495 to 3′-519, amplified together a 644 bp fragment spanning 56 bp of the intron, the prepeptide (86 bp), the protein coding part (399+3) and 100 bp of the 3′ UTR (untranslated region). The same PCR reactions were set up with the 5′ primer biotinylated instead of the 3′ primer. The PCR reactions were subjected to PCR in an Idaho technology PCR machine (Idaho Falls, Id.) with the following PCR profile 30 cycles of 96° C. for 30 seconds, 51° C. for 30 seconds and 74° C. for 1 minute. This profile was followed by 5 minutes at 74° C. 5 μL of the PCR reactions were visualised on an 1% agarose-gel.

DNA Sequencing and Analysis

The remaining PCR-reactions were subjected to DNA sequencing using an Amersham-Pharmacia-Biotech ALF-express DNA sequencer. The 45 μl biotinylated PCR fragments were bound to streptavidin coated combs according to the manufacturers instructions. The DNA strands were separated and the biotinylated 3′ strands were subjected to Sanger-dideoxy-sequencing using Cy5 labelled sequencing primers, T7 DNA polymerase and other components of the Auto Load SPS kit (Amersham Pharamacia Biotech, Uppsala, Sweden). Sequencing from the 5′ end was performed with the primer, 5′-Cy5-tctgcttcttctgttggggcttatg-3′, binding to the DNA sequence coding for the pre-peptide (Pos. 588-612). Sequencing from the 3′ end was performed on fragment biotinylated in the 5′-end. The 3′ sequence primer was 3′-Cy5-gatcttggctatgattgaggagctt-3′ located in the 3′UTR position 5′-1101 to 3′-1 077). The combs were placed in the wells of an acrylamide sequencing gel (ready-gel, AP-Biotech) to release the sequence products.

The sequence gel was run for 700 minutes at 1500 V and subsequently the sequence raw-data was exported from the sequence-program to the evaluation program AlfWin (Amersharn Pharmacia Biotech). The DNA sequences were analysed using the software DNASIS together with studies of the sequence peak-pattern in the analyse program AlfWin.

Approximately 460 bp was analysed by alignment of the sequences sequenced in 5′ and 3′ directions, containing the entire gene coding for the mature protein (399 bp) and some sequence in the pre-peptide and the 3′UTR.

Endonuclease Restriction Digestion

17 μL non-biotinylated PCR reaction was incubated with either 10U Cla I or 10U Pst I in an appropriate digestion buffer (Life Technologies). The samples were digested over night and subsequently analysed on an 1.5% agarose gel containing ethidium bromide.

Results

To ensure that the highly homologous DNA region of the EPX/EDN (RNASE2) gene had not been co-amplified, the 644 bp PCR fragment was subjected to the ECP gene specific Cla I endonuclease digestion. The digestion gave rise to complete digestion (and two bands of sizes 241 and 403) showing that only the region containing the ECP gene had been amplified.

The sequence-analysis of the region on chromosome14 containing the ECP gene of the 70 subjects gave rise to a heterogenous result. Single base substitutions were discovered at position 775, 926 and 1054. The base pair substitution at position 775 gave a shift of amino acid 45 from Arg to Cys (CGT→TGT). The base substitution at position 926 gave a shift of amino acid 97 from Arg to Thr (AGG→ACG). The base substitution at 1054 was located in the 3′UTR. The base substitution at position 775 was present only in a heterozygous form, while the base substitution at position 926 was present as both heterozygous and homozygous forms.

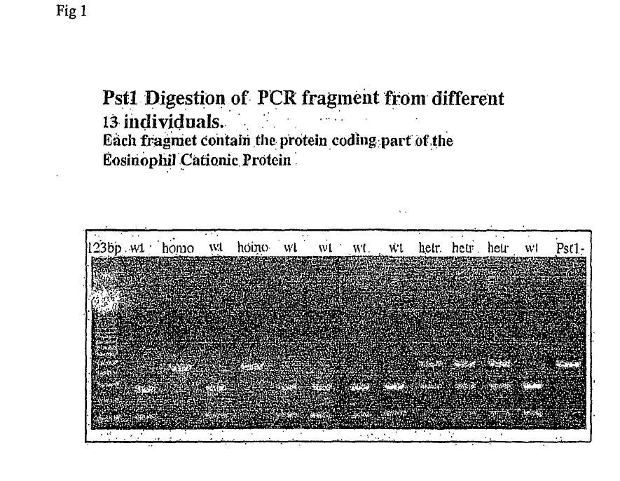

The variant located at position 926 was found to be located at a restriction endonuclease site specific for the enzyme Pst I (CTGCAG changed to CTGCAC). This base-change inhibits the DNA cleaving activity of the enzyme. Therefore the material screened by sequencing were RFLP-analysed by Pst I digestion. The 644 fragment was cleaved into fragments of 213 and 431 bp as shown in FIG. 1. The wild type form gave rise to complete cleavage of the fragment while the homozygous form did not cleave at all. The heterozygous form of the base substitution gave rise to an intermediate, both cleaved and uncleaved fragments (FIG. 1). The result of the cleavages of the 70 individuals was in accordance with the sequencing results.

We have shown in this study that gene variants of ECP do exist. Thus, we found two major base changes, which both gave rise to changes in the amino acid sequence. The change of arginine to threonine at amino acid position 97 seemed to be very common with a prevalence of almost 50% in a population study. Thus, 53% of the subjects investigated had the wild type with arginine in the 97 position and 8% being homozygous with substitution of arginine with threonine. The remaining subjects were heterozygous with respect to the mutation.

### Example 2

### Correlation of the ECP Gene SNP with Asthmatic/Allergic Disorders

Patients

The cross sectional population consisted of 209 medical students, 91 women and 118 men from which blood was taken routinely as part of their course in clinical chemistry. All students filled in a health declaration in which they indicated, among other things, whether they suffered from any chronic disease such as allergy. In 16 students no information about allergy was given. The students were informed about the purpose of the DNA analysis of their blood. 95% of the students gave written informed consent. The atopic status of the medical students was investigated in 117 subjects by Phadiatop (Pharmacia Diagnostics AB, Uppsala, Sweden).

The hospitalized group of patients consisted of 97 patients with suspected asthma. Forty-nine of these patients had a final diagnosis of asthma with atopy and 27 a final diagnosis of asthma without atopy. Twenty-one patients did not fulfill the criteria of asthma according to the criteria of the American Thoracic Society. The patients were informed by the doctor of the purpose of the study and blood for DNA analysis was drawn after oral consent. Atopy was assessed by skin prick test against 10 common allergens and/or by in vitro testing by means of the Pharmacia CAP system. The study was approved by the ethics committee at The Medical Faculty, Uppsala University.

Methods

DNA was prepared from whole blood by a slightly modified method as described (Kawasaki, 1990, PCR Protocols, A Guide to Methods and Protocols, Ed. MA Innis pp 146-152, Academic Press, San Diego) and the ECP gene was amplified by PCR using appropriate primers (described in Example 1). The amplified gene material was subjected to cleavage by the restriction enzyme PstI and the DNA products analyzed by means of agarose electrophoresis. The wild type gene was completely cleaved and gave rise to two distinct bands, whereas the mutated and homozygote gene were left uncleaved giving rise to only one band. The heterozygote gene was a mixture between the wild type gene and the homozygous giving rise to three bands.

Statistics

Fisher's exact test and Chi²-test were used to estimate significant differences between the proportion of the different ECP variants.

Results

The prevalences of the ECP mutation-97 among the medical students were 53% having the wild type variant, 39% the heterozygous variant and 8% the homozygous variant.

Table 1 shows that, among those students who on the questionnaire had indicated that they were allergic, the prevalence of wild type ECP is more common than among those who have not indicated allergy on the questionnaire (p=0.02).

TABLE 1
The prevalence of ECP-mutation 97 among 193 non-allergics
and allergic students (p = 0.02, χ²-test)

| | Non-allergics | Allergics |
|---|---|---|
| Wild type | 47% (67) | 65% (33) |
| Heterozygous | 42% (59) | 35% (18) |
| Homozygous | 11% (16) | 0% |

None of the 16 students who had the homozygote variant indicated allergy on the questionnaire in spite of the fact that about 25% of all students indicated allergy (p=0.02). As shown in table 2 the relation of the ECP variants to allergy was not affected by the subject being atopic or not.

TABLE 2
The prevalence of the ECP-inutation 97 among 117 atopics
and non-atopic students (p = NS, χ²-test)

| | Atopy (Phadiatop +) | Non-atopy (Phadiatop −) |
|---|---|---|
| Wild type | 55% (28) | 52% (34) |
| Heterozygous | 41% (21) | 36% (24) |
| Homozygous | 4% (2) | 12% (8) |

Among the asthmatic patients the prevalences of the ECP variants were similar to the student population with 51% having the wild type, 45% being heterozygous and 4% being homozygous. However, asthmatic subjects with atopy had higher prevalence of the wild type and lower prevalence of the heterozygous+homozygous type than the asthmatics without atopy (table 4), p=0.04 (χ²-test).

Table 3 shows the distribution of the variants and allergy among the students that were Phadiatop-positive. The majority of those students who reported allergy had the genetic wild type variant whereas the majority of those students who reported no allergy were either hetero or homozygous (p=0.0001).

TABLE 3
The prevalence of the ECP-mutation 97 in relation to allergy among
46 Phadiatop-positive students (p = 0.0001, Fisher's exact test)

| | Allergic asthma | Non-allergic asthma |
|---|---|---|
| Wild type | 79% (23) | 18% (3) |
| Homo and heterozygous | 21% (6) | 82% (14) |

Table 4 shows the distribution of the variants among the students that were asthmatic. The majority of those students who reported atopic asthma had the genetic wild type variant whereas the majority of those students who reported non-atopic asthma were either heterozygous or homozygous.

TABLE 4
The prevalence of the ECP-mutation-97 in 76 asthmatic
subjects with and without atopy (p = 0.04, χ²-test)

| | Atopic asthma | Non-atopic asthma |
|---|---|---|
| Wild type | 61% (30) | 41% (9) |
| Heterozygous | 39% (19) | 52% (14) |
| Homozygous | 0 | 7% (2) |

We have shown that the ECP mutation 97 is common in the Swedish population. We have also shown that this mutation is related to the development of allergic manifestations, since allergies had a significantly higher prevalence of the wild type of ECP and no subjects with reported allergic symptoms had the homozygote variant. This data indicates that the change in the amino acid 97 of the ECP molecule may be of major importance for the development of allergic symptoms and points to a central role of this molecule and the eosinophil in these processes.

In the student group we related the genetic variants to self reported symptoms of allergy and although the students all had been studying medicine for three years, the perception of allergy varies between subjects. Therefore it was important to confirm the relationship to allergy in a group of patients with allergic disease in which experienced allergists had made an unbiased diagnosis. In this group of asthmatic subjects, the subjects with atopic asthma had a significantly higher prevalence of the wild type ECP mutation 97 than the non-atopic asthmatics. Overall, however, the prevalence of asthma was not more common among those with the wild type ECP mutation, which indicates that this mutation is not predictive for the development of asthma, but for the development of allergy. This distinction is important and in keeping with the fact that eosinophils are predominantly found at increased numbers in the lungs of atopic asthmatics and that elimination of eosinophils by antibodies to interleukin (IL)-5 (IL-5) or IL-12 had little impact on the bronchial hyper-responsiveness.

The prevalence of the ECP-mutation 97 was not related to the subject being atopic, but when the Phadiatop-positive students were studied separately the impact of the genetic variants for the development of allergic disease became even more striking. Thus, more than 70% of the students who were Phadiatop positive, but had the hetero or homozygous genotype, did not report allergic manifestations. Contrasted by the fact that of those who reported allergic symptoms almost 80% had the wild type variant. Converted into odds ratios the overall risk of developing allergy if you are Phadiatop positive is 117 (15-926) (95% CI), whereas this risk is increased to 240 (31-1900) if you have the wild type variant, but only 13.2 (1.46-121) if you have the hetero or homozygous variant i.e. being atopic and having the wild type variant increases the risk of developing allergic manifestations by about 18 times. Indeed these calculations have to be interpreted cautiously given the fact the relative low numbers on which these are based.

We conclude from this study that mutation-97 of the ECP gene is predictive of a person becoming allergic or not. This finding indicates a pivotal role of the ECP molecule for development of allergic symptoms.

### Example 3

### Recombinant ECP RNase Activity Assay

Material and Methods

Recombinant ECP was produced in the Baculovirus system. One recombinant product was the wild type ECP and the other the ECP mutation 97. The RNase activity of the ECP variants was tested by a commercial kit (RnaseAlert Lab Test Kit, described above) according to the instructions of the manufacturer (Ambion Inc. Texas, USA). The preparations were diluted in RNase buffer to a concentration of 10 ng/ml of ECP as measured by a specific radio immunoassay (Pharmacia Diagnostics, Uppsala, Sweden). Baculovirus supernatants with no expressed ECP were used as controls.

The control preparations contained endogenous RNase activity. This activity was subtracted from the activity of the preparations containing recombinant ECP.

Results

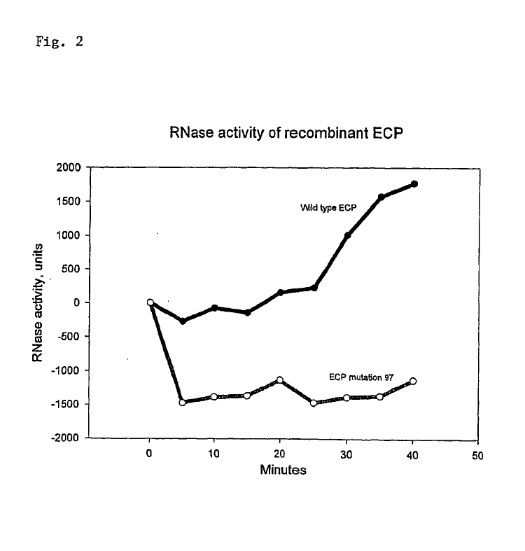

The results are shown in FIG. 2. Wild type recombinant ECP has significant RNase activity, whereas the ECP mutation 97 has no activity. In contrast ECP mutation 97 inhibits the endogenous activity of the protein-rich and RNase containing supernatant. The results are means of 6 experiments with very similar kinetics.

Discussion

The results shown in FIG. 2 confirm that ECP mutation 97 may be used as an effective antagonist of wild-type ECP in the prevention and/or treatment of allergic and/or asthmatic disorders. The assay of Example 3 may be used to identify other such ECP antagonists.

1. Use of a protein selected from;
(a) an ECP,
(b) a mutant of said ECP, and
(c) a fragment of (a) or (b),
said protein lacking an arginine 97 of wild-type ECP or its equivalent, in the preparation of a medicament for the prevention and/or treatment of allergic or asthmatic disorders.

2. Use of a DNA molecule encoding a protein selected from;
(a) an ECP,
(b) a mutant of said ECP, and
(c) a fragment of (a) or (b),
said protein lacking an arginine 97 of wild-type ECP or its equivalent, or of a protein encoded thereby, in the preparation of a medicament for the prevention and/or treatment of allergic or asthmatic disorders.

3. Use according to claim 1 or 2 wherein arginine 97, or its equivalent, is replaced by threonine or a bioisostere thereof.

4. Use of an Eosinophil cationic protein (ECP), or a fragment thereof, comprising a modification at amino acid residue 97 or its equivalent such that residue 97 or its equivalent is any amino acid other than arginine; in the preparation of a medicament for the prevention and/or treatment of allergic or asthmatic disorders.

5. Use of a protein having the amino acid sequence as shown in SEQ I.D. No. 1, or a fragment thereof, wherein residue 97 is any amino acid other than arginine; in the preparation of a medicament for the prevention and/or treatment of allergic or asthmatic disorders.

6. Use according to claims 4 or 5 wherein residue 97 or its equivalent is threonine or a bioisostere thereof.

7. Use according to any of claims 1-6, wherein the protein includes the sequence C T Y corresponding to amino acid residues 96-98 of wild-type ECP, or a biostere thereof.

8. Use according to claim 7, wherein the biostere is of formula C X₁ Y, wherein X₁ is any amino acid residue other than arginine.

9. Use of a protein having SEQ ID No. 4 in the preparation of a medicament for the prevention and/or treatment of allergic or asthmatic disorders.

10. A protein selected from;
(a) an ECP,
(b) a mutant of said ECP, and
(c) a fragment of (a) or (b),
wherein arginine 97 of wild-type ECP, or its equivalent, is replaced by any amino acid other than valine, glycine or lysine.

11. A protein according to claim 10 wherein arginine 97, or its equivalent, is replaced by threonine or a bioisostere thereof

12. An Eosinophil cationic protein (ECP), or fragment thereof, comprising a modification at amino acid residue 97 or its equivalent such that residue 97 or its equivalent is any amino acid other than arginine, valine, glycine or lysine.

13. A protein having the amino acid sequence as shown in SEQ I.D. No. 1, or a fragment thereof, wherein residue 97 is any amino acid other than arginine.

14. A polypeptide according to claims 12 or 13 wherein residue 97 or its equivalent is threonine or a bioisostere thereof

15. A protein according to any of claims 10-14, including the sequence C T Y corresponding to amino acid residues 96-98 of wild-type ECP, or a biostere thereof.

16. A protein according to claim 15, wherein the biostere is of formula C X₁ Y, wherein X₁ is any amino acid residue other than arginine, valine, glycine or lysine.

17. A protein having SEQ ID No. 4.

18. An isolated DNA molecule encoding a protein of any preceding claim.

19. An isolated DNA molecule comprising an ECP-encoding polynucleotide in which guanosine at position 926 or its equivalent is substituted by cytosine.

20. A DNA molecule according to claim 19, having the nucleotide sequence shown as SEQ ID No. 3, or a fragment thereof comprising residue 926.

21. A DNA molecule according to claim 18, having SEQ ID No. 3.

22. Use of a DNA molecule of any of claims 18-21, or of a polypeptide encoded thereby, in the preparation of a medicament for the prevention and/or treatment of allergic or asthmatic disorders.

23. Use of an isolated DNA molecule comprising an ECP-encoding polynucleotide or a fragment thereof in a method of determining in a biological sample the nucleotide residue at position 926 or its equivalent.

24. Use of a pair of oligonucleotide primers which comprise nucleotide sequences flanking position 926, or its equivalent, of an ECP-encoding polynucleotide in a method of determining in a biological sample the nucleotide residue at position 926 or its equivalent.

25. A method of diagnosis of a predisposition to an allergic or asthmatic disorder or an increased likelihood of having such a disorder comprising determining in a biological sample, the presence or absence of a protein as defined in any of claims 10-17 or a nucleotide sequence as defined in any of claims 18-21.

26. A method according to claim 25, using an antibody capable of recognising a said protein.

27. A method according to claim 25, comprising detecting the presence of a G to C polymorphism at position 926 on an ECP encoding polynucleotide.

28. The method according to claim 27, wherein the polymorphism is detected using PCR primers which amplify a DNA segment comprising a nucleotide at position 926 on an ECP encoding polynucleotide.

29. The method according to claim 28, wherein the PCR primers are SEQ ID NO: 5 and SEQ ID NO: 6.

30. A pair of PCR primers which amplify a DNA segment of an ECP encoding polynucleotide, wherein the amplified DNA segment is up to 500 base pairs in length and nucleotide position 926 of the ECP encoding polynucleotide is substantially towards the middle of the DNA segment.

31. A PCR primer, which is selected from the group consisting of SEQ ID Nos: 5 and 6.

32. A method of detecting the presence or absence of WT-ECP-DNA or an ECP encoding nucleotide sequence, comprising making a Pst I digest of a sample believed to contain said WT-ECP-DNA or nucleotide sequence and detecting the fragments produced.

33. An assay for detecting a compound capable of mimicing a protein as defined in any of claims 10-17 or a nucleotide sequence as defined in any of claims 18-21.

34. An assay for detecting a compound capable of antagonising wild-type ECP expression and/or activity.

35. An assay of claim 33 or 34 comprising detecting cytotoxic properties with respect to the killing of bacteria and/or cancer cells.

36. An assay of claim 33 or 34 comprising measuring RNase activity.

37. An assay of claim 33 or 34 comprising measuring the capacity of the compound to affect fibroblasts in their production of proteoglycans.

38. A compound identified by means of the assay of any of claims 33-37.

39. Host cells capable of expressing a protein defined in any of claims 10-17.

40. Host cells according to claim 25, comprising a nucleotide sequence defined in any of claims 18-21.

41. A diagnostic kit comprising the pair of PCR primers according to claim 30.

42. A diagnostic kit comprising an antibody as defined in claim 26.

43. A kit comprising a diagnostic kit according to claim 41 or 42, and a therapeutic agent.

44. A kit according to claim 43, wherein the therapeutic agent is for the treatment of an allergy or asthmatic disorder.

45. A kit according to claim 43 or 44, wherein the therapeutic agent is a compound as defined in claim 38.


SEQUENCE LISTING (6 sequences)

SEQ ID NO: 1 - DNA, 1452 bp, Homo Sapiens
ctgccagcagcgtatagttttcacccagagtccagatcccaccggcaaaactctgtctaacacaggatgacttggaattagagtccgtatagcagaaagagcagcagggctgtccttgggtatccgttgctcagccaagtcatcaaataaaaaggatgattgcacaagtggaccatgtgtcaatctgtgggtttctgcatggccagacccaccaagggaagctttatttaaacagttccaagtaggggagaccagctgcccctgaaccccagaacaaccagctggatcagttctcacaggagccacagctcagagactgggtaagtcaacaatccccagagctgggacaggaggggcagcgacagggcagcacctgagggagaggtgagctgaagttagtgcttaggagatgtggcacactttggggacaggaagaaaaggaaatgcgaccccagagtggcagcagaggggcctgtgggttgagacactatagagtgtgtcataaccgagaccggataggggagtagttacttctcttcttttcttacaggaaacatggttccaaaactgttcacttcccaaatttgtctgcttcttctgttggggcttatgggtgtggagggctcactccatgccagacccccacagtttacgagggctcagtggtttgccatccagcacatcagtctgaacccccctcgatgcaccattgcaatgcgggcaattaacaattatcgatggcgttgcaaaaaccaaaatacttttcttcgtacaacttttgctaatgtagttaatgtttgtggtaaccaaagtatacgctgccctcataacagaactctcaacaattgtcatcggagtagattccgggtgcctttactccactgtgacctcataaatccaggtgcacagaatatttcaaactgcaggtatgcagacagaccaggaaggaggttctatgtagttgcatgtgacaacagagatccacgggattctccacggtatcctgtggttccagttcacctggataccaccatctaagctcctgtatcagcagtcctcatcatcactcatctgccaagctcctcaatcatagccaagatcccatccctccatgtactctgggtatcagcaactgtcctcatcagtctccataccccttcagctttcctgagctgaagtcccttgtgaaccctgcaataaactgctttgcaaattcatctggaagtgtctgtgtgtcttcctcggccgctctgctgtcatttagtgacaatctgctctagagatttgggtttatcatgaatctctccccctcaatatctgaccaaattccttgattcccccatcatccttcatgtgatacctgattccaggcctgccttaaaaaaaaatccaattgagtcaacttagcattggtctccctagccttaatatctcctctaagcaattttccat

SEQ ID NO: 2 - protein, 133 aa, Homo Sapiens
RPPQFTRAQWFAIQHISLNPPRCTIAMRAINNYRWRCKNQNTFLRTTFANVVNVCGNQSIRCPHNRTLNNCHRSRFRVPLLHCDLINPGAQNISNCRYADRPGRRFYVVACDNRDPRDSPRYPVVPVHLDTTI

SEQ ID NO: 3 - DNA, 1452 bp, Artificial Sequence
  Human derived nucleotide sequence
ctgccagcagcgtatagttttcacccagagtccagatcccaccggcaaaactctgtctaacacaggatgacttggaattagagtccgtatagcagaaagagcagcagggctgtccttgggtatccgttgctcagccaagtcatcaaataaaaaggatgattgcacaagtggaccatgtgtcaatctgtgggtttctgcatggccagacccaccaagggaagctttatttaaacagttccaagtaggggagaccagctgcccctgaaccccagaacaaccagctggatcagttctcacaggagccacagctcagagactgggtaagtcaacaatccccagagctgggacaggaggggcagcgacagggcagcacctgagggagaggtgagctgaagttagtgcttaggagatgtggcacactttggggacaggaagaaaaggaaatgcgaccccagagtggcagcagaggggcctgtgggttgagacactatagagtgtgtcataaccgagaccggataggggagtagttacttctcttcttttcttacaggaaacatggttccaaaactgttcacttcccaaatttgtctgcttcttctgttggggcttatgggtgtggagggctcactccatgccagacccccacagtttacgagggctcagtggtttgccatccagcacatcagtctgaacccccctcgatgcaccattgcaatgcgggcaattaacaattatcgatggcgttgcaaaaaccaaaatacttttcttcgtacaacttttgctaatgtagttaatgtttgtggtaaccaaagtatacgctgccctcataacagaactctcaacaattgtcatcggagtagattccgggtgcctttactccactgtgacctcataaatccaggtgcacagaatatttcaaactgcacgtatgcagacagaccaggaaggaggttctatgtagttgcatgtgacaacagagatccacgggattctccacggtatcctgtggttccagttcacctggataccaccatctaagctcctgtatcagcagtcctcatcatcactcatctgccaagctcctcaatcatagccaagatcccatccctccatgtactctgggtatcagcaactgtcctcatcagtctccataccccttcagctttcctgagctgaagtcccttgtgaaccctgcaataaactgctttgcaaattcatctggaagtgtctgtgtgtcttcctcggccgctctgctgtcatttagtgacaatctgctctagagatttgggtttatcatgaatctctccccctcaatatctgaccaaattccttgattcccccatcatccttcatgtgatacctgattccaggcctgccttaaaaaaaaatccaattgagtcaacttagcattggtctccctagccttaatatctcctctaagcaattttccat

SEQ ID NO: 4 - protein, 133 aa, Artificial Sequence
  Human derived polypeptide sequence
RPPQFTRAQWFAIQHISLNPPRCTIAMRAINNYRWRCKNQNTFLRTTFANVVNVCGNQSIRCPHNRTLNNCHRSRFRVPLLHCDLINPGAQNISNCTYADRPGRRFYVVACDNRDPRDSPRYPVVPVHLDTTI

SEQ ID NO: 5 - DNA, 25 bp, Artificial Sequence
  Primer
ggacagttgctgatacccagagtac

SEQ ID NO: 6 - DNA, 25 bp, Artificial Sequence
  Primer
gtgtgtcataaccgagaccggatag